(19) United States
(12) Patent Application Publication
(10) Pub. No.: US 2005/0176135 A1
(43) Pub. Date: Aug. 11, 2005

(54) Cassette for isolation, amplification and identification of DNA or protein and method of use

(76) Inventor: Brian Jones, Crystal Lake, IL (US)

Correspondence Address: MCANDREWS HELD & MALLOY, LTD, 500 WEST MADISON STREET, SUITE 3400, CHICAGO, IL, 60661, US

(21) Appl. No.: 10/773,833

(22) Filed: Feb. 6, 2004

(51) Int. Cl. C12M 1/34

(52) U.S. Cl. 435/287.2; 435/288.5; 435/288.7

(57) ABSTRACT

The present invention is directed to a device for DNA analysis. More particularly, the present invention is directed to a cassette which comprises a first chamber suitable for isolating DNA from a biological sample that is suspected of containing a target DNA, one or more second chambers suitable for amplifying any target DNA found in the sample, a chamber suitable for digesting the target DNA with restriction endonucleases, a medium for separation of the digestion fragments, and a channel connecting the digestion chamber to the separation medium and being of suitable size for transferring at least a portion of the contents of the digestion chamber to the separation medium. Typically, the medium for separation of the digestion fragments is an electrophoretic medium. The cassette of the present invention can also be used for the separation and identification of a protein of interest in a biological sample. It is within the scope of the present invention that the cassette also contains one or more internal waste chambers into which used reagents and biological sample can be directed and stored for disposal with the cassette. It is also within the scope of the present invention that the cassette contains a chamber for storage or receipt of a biological control sample.

### BACKGROUND OF THE INVENTION

The present invention relates to devices for DNA and/or protein analysis. More particularly, the present invention relates to cassettes which are useful for isolating DNA and/or protein from a biological sample suspected of containing a target DNA or a target protein and ultimately identifying whether the DNA or protein in the sample contains the target DNA or target protein. In the case of DNA, the cassette also provides one or more reaction chambers for the amplification of the target DNA to provide it in a detectable amount. The cassettes of the present invention are useful for screening for a plurality of target DNA molecules in a single sample and have a plurality of uses, including diagnostic medicine, and in the identifying the presence of pathogenic organisms or toxic proteins on foods or surfaces.

The conventional way of analyzing the DNA present in a sample of cells involves performing multiple steps using several different bench top instruments in a laboratory setting. First, the DNA must be extracted from the cells in the sample. This is typically done by performing any number of cell lysing procedures that cause the cell walls to break apart and release their intracellular contents. Next, the DNA is typically separated from the rest of the cell contents, as the presence of other cell contents may be undesirable in subsequent steps. To obtain an amount of DNA suitable for characterization, the DNA is amplified, such as by using the polymerase chain reaction (PCR). The resulting amplified DNA products can then be identified by any number of techniques that are well known in the art.

The ability to perform all of these steps in a single miniaturized device has the potential for saving time and expense. Such miniaturized devices would be made much more portable than conventional apparatus. A miniaturized DNA analysis device would also allow the analysis steps to be automated more easily. As a result, assays could be performed by less highly trained personnel than presently required.

Most efforts at fabricating miniaturized DNA analysis devices have focused on silicon as a substrate. For example, a microchip device made out of silicon that performs the steps of cell lysis, PCR amplification, and electrophoretic analysis has been reported. See Larry C. Water, et al., “Microchip Device for Cell Lysis, Multiplex PCR Amplification, and Electrophoretic Sizing,” Anal. Chem., 70:158-162 (1998). Similarly, U.S. Pat. Nos. 5,639,423, 5,646,039, and 5,674,742 each disclose a microfabricated silicon device suited for performing PCR.

Silicon, however, suffers from a number of disadvantages as a substrate material. The cost of fabricating microfluidic devices in silicon can be relatively high. Silicon's high thermal conductivity can make the thermal cycling needed to perform PCR difficult, and silicon's property of being electrically semiconducting can hamper the operation of components that require the maintenance of a high potential difference. Most importantly, the difficulty of bonding multiple layers of silicon together makes it difficult to integrate complex components into the device.

Other attempts at miniaturization also looked at multilayered devices. For example, U.S. Pat. No. 6,544,734, which issued on Apr. 8, 2003 to Broscoe, et al., entitled “Multilayered microfluidic DNA analysis system and method,” substituted a plurality of green-sheet layers for the silicon layers of the prior art. The green-sheet layers are selected from the group consisting of ceramic particles, glass particles, and glass ceramic particles, which unlike the silicon are non-conducting. However, the multiple green-sheet layers must be fastened together by sintering. The raw materials for the green-sheet layers are expensive as is the sintering process. Therefore, it would be desirable to provide a device for the automated analysis of DNA (or protein) that is made from relatively inexpensive starting materials, such as plastic, and that does not require the casting and assembly of a multiplicity of layers, such as associated with the multi-layered silicon or sintered devices of the prior art.

### BRIEF SUMMARY OF THE INVENTION

The Applicant has discovered that he could make a device suitable for the automated analysis of DNA (or protein) from two opposing plates of molded plastic that are adhered together. More specifically, the Applicant discovered how to make a small disposable device in cassette form that is capable isolating the DNA in a biological sample, amplifying the target DNA if present, and identifying the presence (or absence) of the target DNA in the biological sample. In its simplest form, the device of the present invention comprises a plastic cassette suitable for DNA analysis comprising a top plate and an opposing bottom plate affixed thereto, the plates in combination forming therebetween an isolation chamber suitable for isolating DNA from the biological sample suspected of containing the target DNA, one or more reaction chambers in fluid communication with the isolation chamber and suitable for amplifying any target DNA found in the isolation chamber, a digestion chamber which is the same as or in fluid communication with the amplification chamber and suitable for digesting the amplified target DNA (amplicons) with one or more restriction endonucleases to produce digestion fragments that are characteristic for the target DNA. At this point, the digested amplicons could be removed from the digestion chamber for separation and further analysis. In a more preferred embodiment, the device of the present invention includes a separation chamber in fluid communication with the digestion chamber for receiving and separating the digestion fragments. In the above described embodiments, one of the plates (typically, the top plate) has a port for receiving a biological sample suspected of containing a target DNA, the port being in fluid communication with the isolation chamber. It also has a port in fluid communication with the digestion and/or amplification chambers for receiving amplification and digestion reagents. The latter port is sufficiently sized for receiving a probe capable of withdrawing the reaction mixture containing the digested amplicons, if present.

By the term “in fluid communication” as used herein is meant that there is a path by which fluid, if present, could travel between the two components that are in fluid communication. Typically, fluid communication is achieved by a channel that is molded into one of the plastic plates or both, or it could be a corresponding groove in the face of both plates that forms a channel when opposing faces of the two plates are mated together. It is also within the scope of the term “in fluid communication” that the channel also includes a valve suitable for stopping or allowing the flow of fluid through the channel.

It is preferred that the cassette of the present invention further includes a waste chamber in fluid communication with the isolation chamber and suitable for receiving undesired cellular components, unused sample or reagents or all of the above. Typically, the waste chamber is in fluid communication with the isolation chamber by a first channel. Preferably, the first channel has a first valve operatively positioned therein. By the term “operatively positioned” is meant that the valve is capable of allowing or preventing the flow of liquid in the channel in which it is positioned.

In use, the isolation chamber in the cassette is filled by the injection of sample and reagents under positive pressure. Preferably, the isolation chamber is associated with a piston suitable for drawing fluid into the chamber as the piston is retracted. The distal end of the piston comprises one wall of the isolation chamber. In use, the piston would be retracted to create a volume within the chamber that corresponds to the size of the sample and/or reagents being injected.

In one embodiment of the cassette of the present invention, the isolation chamber is in fluid communication with the one or more reaction chambers by a second channel that is molded into the top plate, or bottom plate or both. Preferably, the second channel has a second valve operatively positioned therein. The number of reaction chambers that can be in fluid communication with a single isolation chamber is dependent upon the size of the sample being processed. Typically, 1 to 24 reaction chambers are split off in parallel or series from a single isolation chamber. Because each reaction chamber is controlled by its own dedicated second valve, the above described cassette of the present invention is capable of running from one to ten reactions simultaneously on aliquots of isolated DNA from the same sample of biological fluid. The number of reaction chambers that receive an aliquot of sample containing isolated DNA from the isolation chamber is a function of the number of valves that open to allow fluid to enter. Thus, a cassette having 24 reaction chambers can run from 1 to 24 distinct reactions on the same sample of isolated DNA. Such a capability is particularly useful when screening patient DNA for a number of genetic disorders, or for identifying bacteria in foods and on various surfaces. To use the multi-screening capabilities of the cassette, each reaction chamber containing an aliquot of liquid from the isolation chamber would be provided with a polymerase enzyme and the appropriate primers for the target DNA to be amplified. By introducing different primers in each reaction chamber, a plurality of different targets can be screened in a single cassette on the same sample.

The reaction chamber suitable for amplifying any target DNA is also capable of functioning as the digestion chamber suitable for specifically digesting the target DNA into shorter DNA fragments that can be separated and screened. This chamber is then in fluid communication with a separation chamber by a third channel. To control the flow of fluid from the reaction/digestion chamber, the third channel has a third valve operatively positioned therein. Each reaction/digestion chamber has its own dedicated third channel that goes to a dedicated point on the separation chamber. The separation chamber has a separation medium therein. Typically, the separation medium is an electrophoretic medium. More typically, the electrophoretic medium is a slab of gel or in a capillary. Suitable electrophoretic mediums are well known in the art and include polyacrylamide gel.

In order to detect the DNA fragments that are formed by digesting the amplified DNA with one or more restriction endonucleases, the fragments are bound to a fluorescent label. As a result, at least a portion of the separation chamber is sufficiently transparent for detecting separated restriction fragments therein. Preferably the entire top plate, or bottom plate or both are transparent and molded from the same plastic. In one embodiment, the sufficiently transparent portion of the separation chamber is sufficiently transparent to visible light. In a preferred embodiment, the sufficiently transparent portion of the separation chamber is sufficiently transparent to ultra-violet light.

By the term “sufficiently transparent to ultra-violet light” is meant that the separation chamber is sufficiently transparent to certain useable wavelengths of UV light in the range of 5 nm to 500 nm to allow one to monitor the fluorescence of any labels attached to the target DNA or target protein. Typically, the chamber is about 50% transmissible to UV light at the wavelength of interest, preferably about 80% transmissible to UV light; more preferably about 95% transmissible to UV light; even more preferably, about 97% transmissible to UV light.

Any plastic can be used to form the cassette of the present invention provided that it is resistant to the chemicals used in the standard separation, amplification and restriction endonucleases digestions, and provided that it is non fluorescent and sufficiently transparent to visible and UV light. A preferred plastic is an acrylic, more preferably a polymethylmethacrylate.

In making the cassette of the present invention, the top plate and the opposing bottom plate are molded and mateable with one another. In one embodiment, the chambers and channels are machined onto one or more faces of the molded plastic plates. Preferably, all of the chambers and channels are molded onto the mating faces of the top plate and the bottom plate. In some embodiments, the channels are molded into one face of the top plate or bottom plate. When the plastic is an acrylic, it is preferred that the top plate and the bottom plate are injection molded.

In the cassette of the present invention, any valve capable of operably controlling the transmission of fluid in the channel may be used. A preferred valve is a compression valve. It has been discovered that a simple compression valve made of elastomer may be used. Suitable elastomers are natural or synthetic rubbers that are sufficiently soft to be able to be conform to the shape of the channel when they are compressed into the channel. When the cassette has a plurality of valves in proximity (See FIG. 7), a valve cluster can be used. In this embodiment, the valve cluster is a strip of elastomer that covers each of the valve positions over the respective channels. The valve closes the channel when a pin pushes the valve into the channel, thereby occluding the channel. Thus, in this embodiment, all valves in the device are by default in the open position and are only closed when external compression is applied to the valve from the outside.

In one embodiment, the strip of elastomer is positioned between the top plate and the opposing bottom plate. In another embodiment, a valve cluster, comprising a strip of elastomer is adhered via injection molding to the outside face of the top plate or bottom plate of the cassette. A hole (valve port) extending between the top and bottom surfaces of one of the plates connects one face of the adhered elastomeric rubber to the channel below and allows a pin to compress the elastomeric rubber down the hole (valve port) and across the channel to completely occlude the channel.

It is also within the scope of the present invention that one or both plates of the cassette of the present invention further comprises a thin elastomeric layer or coating positioned along the edge of the chambers and channels to provide a watertight seal when the surfaces of the top plate and the bottom plate are mated and there is liquid therein. In this embodiment, the layer is typically about 0.010 to 0.090 inches thick; more typically about 0.030 to 0.70 inches; most typically about 0.050 inches thick. In this embodiment, the elastomeric layer provides a compression seal when the surfaces are mated together and allows for minor defects in the molding of the plates.

In the cassette of the present invention, the upper plate is affixed to the lower plate by an adhesive or by ultrasonic welding.

In one embodiment of the cassette of the present invention, the 1 to 24 parallel digestion chambers are each connected by a separate (third) channel to their own sample loading port on a gel slab that is positioned in the separation chamber between the top plate and the opposing bottom plate, respectively. In another embodiment of the cassette of the present invention, the 1 to 24 parallel digestion chambers are each connected by a separate (third) channel to a loading port on 1 to 24 parallel separation chambers. In one embodiment, the 1 to 24 parallel separation chambers are 1 to 24 parallel capillary gel chambers, respectively.

In another embodiment of the cassette of the present invention, both the isolation chamber and the mixing chamber have a unique piston or plunger moveably sealed therein for drawing fluid therein or pushing fluid thereout or both. See FIG. 1. Preferably, the isolation chamber and the mixing chamber are cylindrical and sized so that a plunger tip from a LUER® insulin syringe (1 cc) can be used as the piston or plunger.

### BRIEF DESCRIPTION OF SEVERAL VIEWS OF THE DRAWINGS

FIG. 1 discloses a schematic of a liquid flow system 10, including channels and chambers, of one embodiment of the cassette of the present invention. In this embodiment, the schematic shows 4 reaction (amplification/digestion chambers) although one or more could be present.

FIG. 2 discloses a schematic of a liquid flow system 20, including channels and chambers, of a second embodiment of the cassette of the present invention. Unlike the embodiment in FIG. 1, the embodiment of FIG. 2 has individual separation chambers 29 for each reaction chamber 7. The isolation chamber 5 and the mixing chamber 6 are shown with pistons 27 and 25, respectively, for intaking sample and reagents and for mixing them.

FIG. 3 discloses a cassette 30 of the present invention having 10 amplification/digestion (reaction) chambers that allow for the amplification of 10 different (or the same) DNA targets from a single biological sample. The separation chamber contains a gel slab 9 which has sample wells 32 for introducing an amplified and digested DNA sample from each reaction well.

FIG. 4 discloses a schematic of another flow system 40 utilized in an embodiment of the cassette of the present invention. This schematic is analogous to that shown in FIG. 2, except that the present schematic provides for two additional chambers for sample processing and DNA or protein isolation.

FIG. 5 discloses a cassette of the invention embodying the flow system of FIG. 4.

FIG. 6 discloses another embodiment of the cassette of FIG. 5 having cavities 61 which allow one to monitor the reaction in each of reaction chambers 7 by measuring the fluorescence reaching each cavity 61 when the solution in the reaction chambers is stimulated with an excitation frequency or detected spectrophotometrically, such as by real time PCR.

FIG. 7 discloses the flow system of FIG. 1 having wherein a strip of elastomer 71 over the channels which functions as a valve array (for valves 13 and 14) at the points where the strip crosses the channel. Likewise, elastomeric strip 72, which is dogbone shaped, provides valves 11 and 12 at the heads of the dogbone.

### DETAILED DESCRIPTION OF THE INVENTION

The present invention is directed to a small disposable cassette for DNA and/or protein analysis. The cassette of the present invention has several embodiments that are useful for separating and amplifying DNA, or separating protein from a biological sample. In one embodiment, the present invention is directed to a cassette that has reaction chambers and channels for separating DNA from cells in a biological sample, and amplifying the target DNA if present, for further analysis. Preferably, the device of the present invention also provides a chamber having a medium for identifying the presence (or absence) of the target DNA or protein in the biological sample. In its simplest form, the device of the present invention comprises a plastic cassette suitable for DNA analysis comprising a top plate and an opposing bottom plate affixed thereto, the plates in combination forming therebetween an isolation chamber suitable for isolating DNA from the biological sample suspected of containing the target DNA, one or more mixing and/or reaction chambers in fluid communication with the isolation chamber and having a port for receiving reagents, said reaction chamber suitable for amplifying any target DNA found in the isolation chamber, and a digestion chamber which is the same as or in fluid communication with the amplification chamber and suitable for digesting the amplified target DNA with one or more restriction endonucleases to produce specific digestion fragments that are characteristic for the target DNA. At this point, the digestion fragments could be removed from the digestion chamber of the cassette for separate analysis. Preferably, the cassette also contains a separation chamber in fluid communication with the digestion chamber for receiving and separating the digestion fragments.

In the cassette of the present invention, one of the plates (typically, the top plate) has a port for receiving a biological sample suspected of containing a target DNA. This port is in fluid communication with the isolation chamber. Preferably, the port is positioned above the channel outside the isolation chamber. Typically, the port has a diameter within the range of 0.020 inches to 0.040 inches, more typically the diameter is about 0.030 inches.

The cassette of the present invention is also suitable for analyzing for the presence of a target protein. Since proteins are generally present in greater concentrations than DNA, the chambers utilized for amplification of DNA can be used for reduction of the protein to determine the presence of subunits, or the digestion with a specific enzyme to determine the weight of the fragments produced. As will be pointed out below, the cassette is also constructed to allow for the detection of weak fluorescent signals produced by DNA. Thus, while the best use of the cassette is for the detection of target DNA in a biological sample, it is equally capable of being used to detect the presence of a target protein.

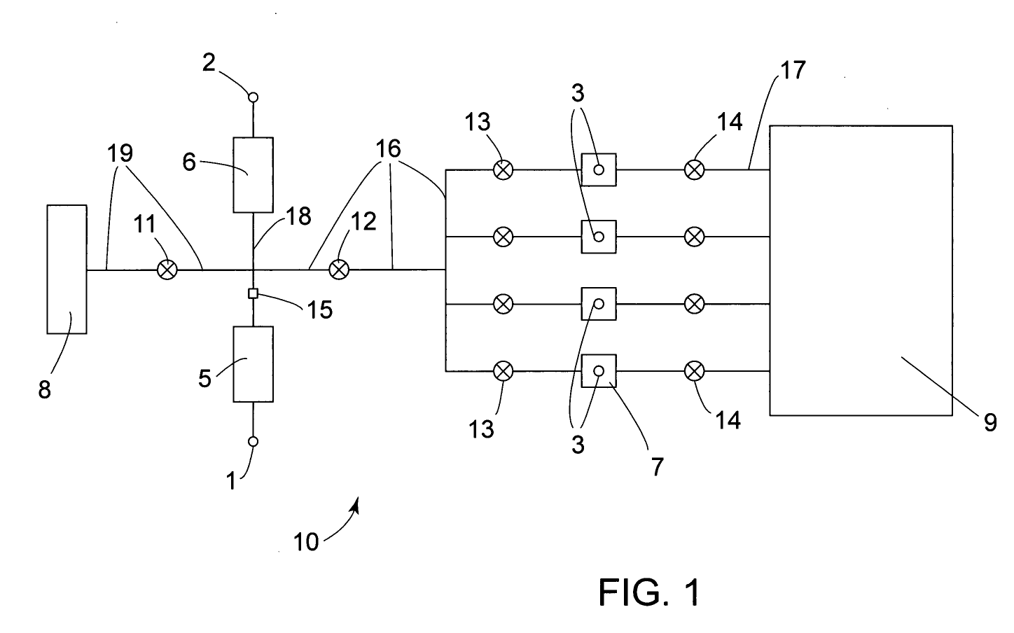

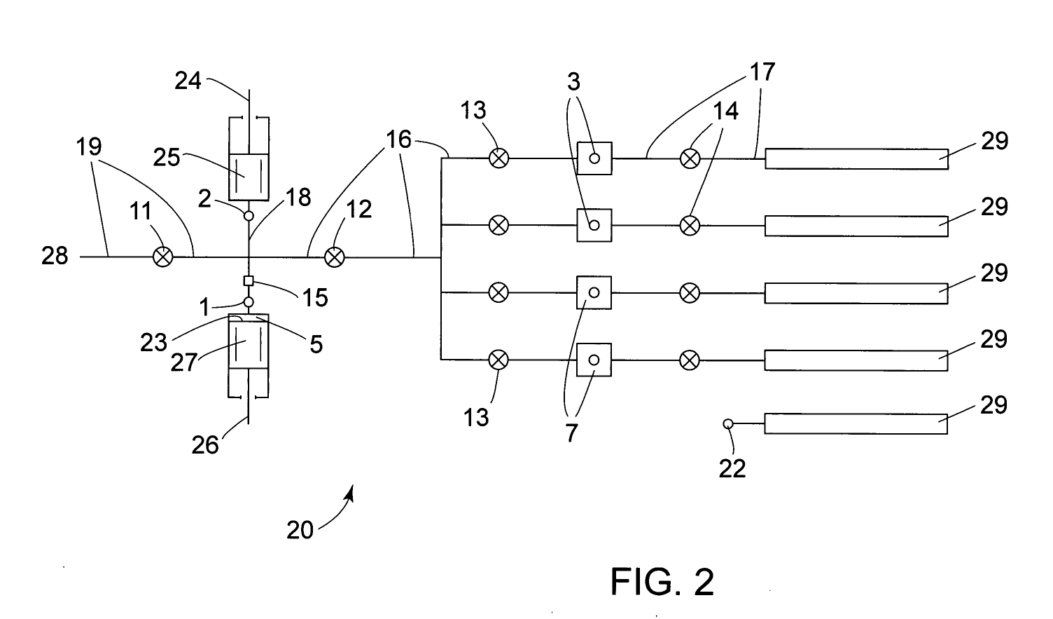

In one embodiment of the present invention, the cassette expels waste through an outlet. See outlet 28 of FIG. 2. It is preferred that the cassette of the present invention further include a waste chamber, such as shown in FIG. 1, in fluid communication with the isolation chamber and suitable for receiving used sample or reagents or both. Typically, the waste chamber is in fluid communication with the isolation chamber by a first channel. Preferably, the first channel has a first valve operatively positioned therein. By the term “operatively positioned” is meant that the valve is capable of allowing or preventing the flow of liquid in the channel in which it is positioned.

The isolation chamber in the cassette is capable of being filled by the injection of sample and reagents under positive pressure. In one embodiment, fluid is transferred from an isolation chamber to a mixing chamber by applying positive pressure through a port at one end of the isolation chamber. Preferably, the isolation chamber is associated with a piston suitable for drawing fluid into the isolation chamber or the mixing chamber as the piston is retracted and expelling precise volumes of liquid as the piston is compressed. In this embodiment, the distal end of the piston provides one wall of the isolation chamber. In use, the piston is retracted to create a volume within the chamber that corresponds to the size of the sample and/or reagents being injected into the chamber.

Because DNA is an intracellular material, a biological sample consists of a DNA extract, at least one nucleated cell, or at least one mitochondria. Typically, the source of DNA is a DNA extract or a plurality of nucleated cells. A typical volume of a biological sample comprises about 1 microliter to about 150 microliters of a biological sample. In screening a patient (e.g., a human) for one or more genetic diseases, an aliquot of a DNA extract from a patient, or a patient's plasma containing the white cell fraction is a suitable specimen for screening the patient's DNA. In the embodiment of FIGS. 1 and 2, the patient sample containing the DNA or cells is injected into the isolation chamber. From the isolation chamber, the sample containing the cells is transferred into a mixing chamber having a port open to the outside for receiving one or more reagents or diluent. When the sample is cellular, a filter is optionally positioned in a channel connecting the isolation chamber with the mixing chamber and having a sufficiently small pore size to retain cells but allowing the fluid to pass through.

By way of example, platelets have a mean diameter of 2.6 microns to 2.9 microns. Lymphocytes have a mean diameter of 10.8 microns to 12 microns, depending upon the cell type. Thus, when the cellular specimen is a platelet, or preferably, a lymphocyte, the filter would typically have a pore size with a diameter in the range of 0.2 microns to 2.0 microns.

Suitable filters are made from nylon and have a pore size ranging from about 0.2 microns to about 2 microns. A suitable nylon filter material having a pore size of 1 micron is commercially available under the trade name NITEX® 03-11 from Sefar America of DePew N.Y.

In those embodiments having a filter, any cellular material is held up at the filter while the carrier solution for the specimen is transferred from the isolation (receiving) chamber to the mixing chamber. The liquid in the mixing chamber is capable of being expelled to waste, either outside the cassette, or preferably to a waste chamber in fluid communication with the mixing chamber. This would leave the cellular material at the filter but free from its carrier fluid.

In use, a solution of cellular lysing agent is added to the biological sample in the cassette to lyse the cells and release the DNA. Lysing solutions are well known in the art and typically are hypotonic solutions that cause the cells to burst. The lysing solutions can also contain enzymes, such as lysozme, that enhance the lysing process. The solution containing the lysed cells is expelled from the isolation chamber, through a filter in the first channel, into the mixing chamber. Preferably, the transfer of fluid from the isolation chamber to mixing chamber is performed by the compression of a piston in the isolation chamber to a predetermined amount and the withdrawal of the piston a corresponding amount in the mixing chamber. This piston and filter arrangement is shown in FIG. 2. The cellular DNA, including the target DNA, is now in the lysing solution in the mixing chamber. The DNA solution from the mixing chamber is transferred back to the isolation chamber.

The DNA solution in the isolation chamber can be further purified using standard isolation techniques, such as by injecting paramagnetic beads in a hypertonic saline solution into the isolation chamber. Under these conditions, the DNA binds to the magnetic beads. If the biological sample that was initially injected into the isolation chamber was a DNA extract, rather than cells, then the paramagnetic beads in a hypertonic saline solution are injected into the isolation chamber directly with the DNA extract.

The isolation chamber is in fluid communication with the one or more reaction chambers by a second channel that is molded into the top plate, or bottom plate or both. Preferably, the second channel has a second valve operatively positioned therein. The number of reaction chambers that can be in fluid communication with a single isolation chamber is dependent upon the size of the sample being processed in the isolation chamber. Typically, 1 to 24 reaction chambers are split off in parallel or series from a single isolation chamber. Because each channel going to a reaction chamber is controlled by its own dedicated second valve, the device is capable of running from one to twenty-four reactions simultaneously on the same or different sized aliquots of isolated DNA obtained from a single sample of biological fluid. The number of reaction chambers that receive an aliquot of sample containing isolated DNA from the isolation chamber is a function of the number of valves that are opened (typically, sequentially) to allow fluid to enter each reaction chamber. Thus, a cassette having 24 reaction chambers can run from 1 to 24 distinct reactions on the same sample of isolated DNA. Such a capability is particularly useful when screening patient DNA for a number of genetic disorders, or for identifying bacteria in foods and on various surfaces. To use the multi-screening capabilities of the cassette, each reaction chamber is provided with an aliquot of liquid from the isolation chamber and is further provided with a polymerase enzyme and the appropriate primer(s) for the target DNA to be amplified. By introducing different primers in each reaction chamber, a plurality of different targets (if present) can be amplified and specifically digested by restriction endonucleases in a single cassette on the same sample. In a preferred embodiment, the cassette also contains a separation chamber that Preferably, the samples of amplicons are screened

Amplification of DNA by the polymerase chain reaction (PCR) using a thermostable DNA polymerase, deoxyribonucleoside-5′-triphosphates, and a pair of oligonucleotide primers is well-known in the art. See e.g., U.S. Pat. No. 4,683,195 to Mullis et al., which issued on Jul. 28, 1987, and is entitled “Process for amplifying, detecting, and/or-cloning nucleic acid sequences;” U.S. Pat. No. 4,683,202 to Mullis et al., which issued on Jul. 28, 1987, and is entitled “Process for amplifying nucleic acid sequences;” U.S. Pat. No. 4,800,159 to Mullis et al., which issued on Jan. 24, 1989, and is entitled “Process for amplifying, detecting, and/or-cloning nucleic acid sequences;” U.S. Pat. No. 4,889,818, which issued on Dec. 26, 1989 to Gelfand, et al., entitled “Purified thermostable enzyme;” and U.S. Pat. No. 4,965,188 to Mullis et al., which issued on Oct. 23, 1990, and is entitled “Process for amplifying, detecting, and/or-cloning nucleic acid sequences using a thermostable enzyme.” These references are incorporated herein by reference in their entirety.

PCR is achieved by temperature cycling of the sample, causing DNA to denature (separate), specific primers to attach (anneal to the template DNA), and replication (primer extension) to occur. One cycle of PCR is usually performed in 2 to 8 min, requiring 1 to 4 hours for a 30-cycle amplification. The sample temperature response in most PCR instrumentation is very slow compared to the times required for denaturation, annealing, and extension. The physical (denaturation and annealing) and enzymatic (extension) reactions in PCR occur very quickly. Amplification times for PCR can be reduced from hours to less than 15 min. The following individual applications, which disclose such a rapid cycling system are incorporated herein by reference in their entireties: U.S. application Ser. No. 08/818,267, filed Mar. 17, 1997, entitled “Method for Detecting the Factor V Leiden Mutation,” which is a continuation-in-part of U.S. patent application Ser. No. 08/658,993, filed Jun. 4, 1996, entitled “System And Method For Monitoring PCR Processes,” which is a continuation-in-part of U.S. patent application Ser. No. 08/537,612, filed Oct. 2, 1995, entitled “Method For Rapid Thermal Cycling of Biological Samples,” which is a continuation-in-part of U.S. patent application Ser. No. 08/179,969, filed Jan. 10, 1994, (now U.S. Pat. No. 5,455,175), entitled “Rapid Thermal Cycling Device,” which is a continuation-in-part of U.S. patent application Ser. No. 07/815,966 filed Jan. 2, 1992, (now abandoned) entitled “Rapid Thermal Cycling Device” which is a continuation-in-part of U.S. patent application Ser. No. 07/534,029 filed Jun. 4, 1990, (now abandoned) entitled “Automated Polymerase Chain Reaction Device.” The copending U.S. application filed in the U.S. Patent and Trademark Office on Jun. 4, 1997, entitled “System and Method for Carrying Out and Monitoring Biological Processes” as Ser. No. 08/869,275 and naming Carl T. Wittwer, Kirk M. Ririe, Randy P. Rasmussen, and David R. Hillyard as applicants, is also hereby incorporated by reference in its entirety. Rapid cycling techniques are made possible by the rapid temperature response and temperature homogeneity possible for samples in high surface area-to-volume sample containers. For further information, see also: C. T. Wittwer, G. B. Reed, and K. M. Ririe, “Rapid cycle DNA amplification,” in K. B. Mullis, F. Ferre, and R. A. Gibbs, “The Polymerase Chain Reaction, Birkhauser, Boston, 174-181, (1994).

When the polymerase chain reaction is being utilized, the rapid alternating heating and cooling steps are performed in the amplification chamber using an external Peltier heater and cooler, such as available from Marlow Industries.

Alternatively, DNA amplification is performed in the amplification chamber using a thermostable ligase, such as disclosed in U.S. Pat. No. 6,054,564 which issued on Apr. 25, 2000 to Barany et al., and entitled “Thermostable ligase mediated DNA amplification system for the detection of genetic diseases,” which is incorporated herein by reference in its entirety.

Preferably, DNA amplification is performed in the amplification chamber at room temperature without thermocycling, using a DNA polymerase. Such a method is disclosed in U.S. patent application Ser. No. 10/125,973 by Benkovic and Salinas, filed on Apr. 19, 2002, and entitled “Methods for nucleic acid manipulation,” and having Publication Number 20030143525, which is incorporated herein by reference in its entirety. Basically, the method of amplifying a target nucleic acid of the '973 application comprises a) reacting a nucleic acid duplex with a primer that is complementary to a target sequence within a nucleic acid duplex, in the presence of a recombination factor, such as bacteriophage T4 UvsX protein, E. coli Rec A protein, or Rad51, to form a recombination intermediate, without previously denaturing the nucleic acid duplex; and b) admixing a polymerase enzyme, a clamp protein, and a clamp loader protein, with the recombination intermediate to form a polymerase complex, whereby the polymerase replicates the target sequence.

The reaction (amplification) chamber in which the target DNA is amplified is also capable of functioning as a digestion chamber suitable for digesting the amplified target DNA (amplicons) with one or more restriction endonucleases into smaller DNA fragments that can be separated and screened The use of restriction endonucleases to cleave a DNA molecule at specific points in the chain is well known in the art. The use of the resulting DNA fragments to identify the source of the original DNA molecule is also well known in the art. For example U.S. Pat. No. 6,495,325, which issued on Dec. 2, 2002 to van Haeringen, et al. and is entitled “Detection and quantification of micro-organisms using amplification and restriction enzyme analysis,” is incorporated herein by reference in its entirety. A large variety of restriction endonucleases that cleave DNA molecules at enzyme specific restriction sites are well known in the art and commercially available by catalog from sources such as CHIMERx in Madison Wis.

The reaction/digestion chamber is in fluid communication with the separation chamber by a third channel. To control the flow of fluid from the reaction/digestion chamber, the third channel has a third valve operatively positioned therein. Each reaction/digestion chamber has its own dedicated third channel that goes to a dedicated point on the separation chamber. The separation chamber has a separation medium therein. Typically, the separation medium is an electrophoretic medium. More typically, the electrophoretic medium is a slab of gel or a capillary containing an electrophoretic medium. Suitable electrophoretic mediums and their use are well known in the art and include a polyacrylamide gel, agarose gel or a combination thereof in slabs or in capillary devices.

In order to effect the electrophoretic separation, two opposite ends of the gels are exposed to an electrically conducting buffer which is connected by electrodes, typically carbon or platinum, to an electric power source. Once the electrical power source is switched on, the electric field forces negatively charged molecules to move towards the anode and positively charged molecule to move towards the cathode.

DNA is negatively charged and therefore, in agarose or acrylamide gels which provide sieving action, DNA molecules move towards the anode (+) at a rate which depends on their size, wherein the smaller the molecules the faster they move, and their charge.

In the electrophoretic separation of proteins, the proteins are often treated with an ionic detergent, such as sodium dodecylsulphate (SDS). The negatively charged dodecylsulphate anions interact with hydrophobic domains on the protein molecules, thus creating negatively charged protein/SDS complexes that undergoing electrophoresis separation similar to DNA molecules by moving toward the anode.

A typical electrophoretic gel is comprises agarose in a 1× Tris-borate-EDTA (TBE) buffer, pH 7.5. However, bufferless gels are also suitable for use in the separation chamber of the present invention. The making and use of such bufferless gels is disclosed in U.S. Pat. No. 5,209,831, which issued on May 11, 1993 to MacConnel, the contents of which are hereby incorporated by reference as if recited in full herein. MacConnel describes a bufferless disposable cassette having open ends and conductive film electrodes. Another U.S. patent that discloses the use of bufferless gel systems is U.S. Pat. No. 6,569,306, which issued to Read, et al., on May 27, 2003 and is entitled “Cassette for gel electrophoresis having solid buffer reservoirs,” the contents of which are hereby incorporated by reference as if recited in full herein. Yet another U.S. patent that discloses a bufferless system is U.S. Pat. No. 6,379,516, which issued to Cabilly, et al. on Apr. 30, 2002, the contents of which are hereby incorporated by reference as if recited in full herein. In addition to a bufferless gel, Cabilly discloses a cation exchange matrix in contact with the anode and with the gel matrix, wherein ions released from the anode (e.g., copper) are exchangeable with ions released from the body of the cation exchange matrix.

Suitable shapes and sizes of wells on the electrophoretic gels are well known in the art. Typically, the wells are substantially rectangular in shape as shown to provide a sizeable target for loading the gel yet provide a substantial surface area on the “starting line” of the gel.

In order to detect the DNA restriction fragments that are formed by digesting the amplified target DNA with one or more restriction endonucleases, the fragments are bound to a fluorescent label. The use of ethidium bromide or fluorescent labels is well known in the art. However, any signal produced by the labeled DNA is relatively weak compared to the signals obtained from a labeled protein. Therefore, at least a portion of the separation chamber is sufficiently transparent for detecting the separated restriction fragments therein. Preferably the entire top plate of the cassette, or bottom plate or both are transparent and molded from the same plastic. In one embodiment, the sufficiently transparent portion of the separation chamber is sufficiently transparent to visible light. In a preferred embodiment, the sufficiently transparent portion of the separation chamber is sufficiently transparent to ultra-violet light.

By the term “sufficiently transparent to ultra-violet light” is meant that the separation chamber is sufficiently transparent to certain useable wavelengths of UV light in the range of 5 nm to 500 nm to allow one to monitor the fluorescence of any labels attached to the target DNA or target protein. Typically, the chamber is about 50% transmissible to UV light at the wavelength of interest, preferably about 80% transmissible to UV light; more preferably about 95% transmissible to UV light; even more preferably, about 97% transmissible to UV light.

Any plastic can be used to form the cassette of the present invention provided that it is resistant to the chemicals used in the standard separation, amplification and restriction digestion reactions, and provided that it is non fluorescent and sufficiently transparent to visible and UV light. A preferred plastic is an acrylic, more preferably a polymethylmethacrylate. An especially preferred UV transparent polymethylmethacrylate is PLEXIGLAS® V920-UVT which is commercially available from Atoglas, which is a subsidiary of ATOFINA Chemicals, Philadelphia Pa.

In making the cassette of the present invention, the top plate and the opposing bottom plate are molded and mateable with one another. In one embodiment, the chambers and channels are machined onto one or both mating faces of the molded plastic plates. Preferably, all of the chambers and channels are molded into the mating faces of the top plate and the bottom plate. In some embodiments, the channels are molded into one face of the top plate or the bottom plate. When the plastic is an acrylic, it is preferred that the top plate and the bottom plate are injection molded.

In the cassette of the present invention, any valve capable of operably controlling the transmission of fluid in the channel may be used. A preferred valve is a compression valve. It has been discovered that a simple compression valve made of elastomer may be used. Suitable elastomers are natural or synthetic rubbers that are sufficiently soft to be able to be conform to the shape of the channel when they are compressed into the channel. An elastomer with a hardness of 40 on the Shore A scale is sufficient. Such elastomers include block type copolymers, particularly those based upon styrene-ethylene-butylene-styrene/styrene-ethylene-propylene-styrene (SEBS/SEPS). An especially preferred SEBS/SEPS elastomer is commercially available from MultiBase Inc, of Copley, Ohio under the trade name Multi-Flex® TEA 3405 T1 Trans.

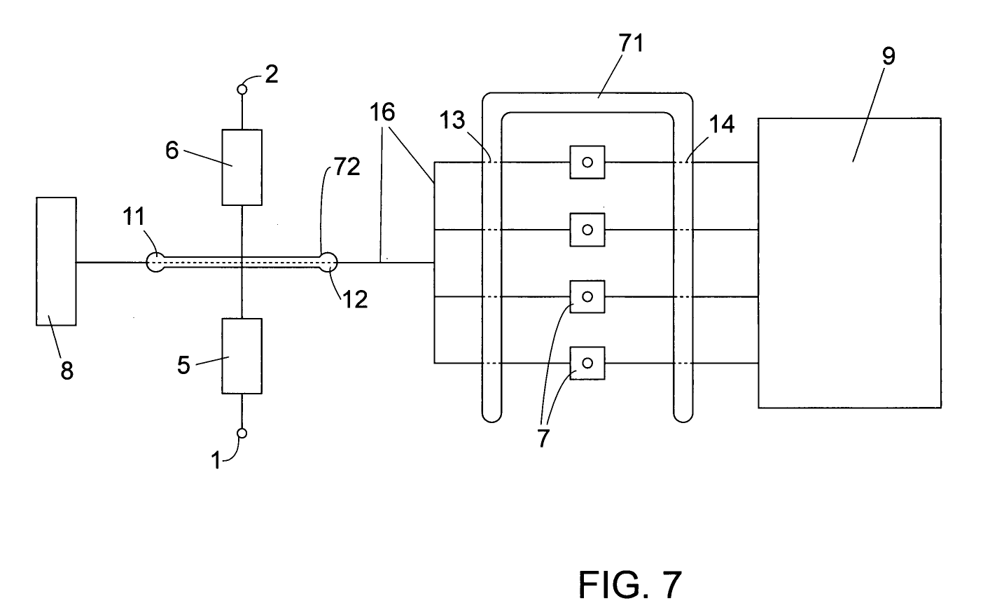

When the cassette of the present invention has a plurality of valves in proximity (See FIG. 7), a valve cluster is used. In this embodiment, the valve cluster is merely a strip of the elastomer shaped and sized to cover each of the valve positions over the respective channels. (See elements 71 and 72 of FIG. 7.) The valve closes the channel when a pin enters a hole in the plate and pushes the elastomeric material into the channel, thereby occluding the channel. Thus, in this embodiment, all valves in the device are in the open position and are only closed when external compression is applied to the valve from the outside.

In one embodiment, the valve cluster comprises a layer of elastomeric rubber positioned between the top plate and the opposing bottom plate. In another embodiment, a valve cluster, comprising a layer of elastomeric rubber, is adhered by injection molding to the outside face of the top plate or bottom plate of the cassette. A hole (valve port) between the top and bottom surfaces of the plate connects one face of the elastomeric rubber to the channel below and allows a pin to compress the elastomeric rubber down the hole (valve port) and across the channel to completely occlude the channel.

It is also within the scope of the present invention that the cassette further comprising a thin elastomeric layer or coating positioned along the edge of the chambers and channels to provide a watertight seal when the surfaces are mated and there is liquid therein. The elastomeric layer or coating is typically from 0.010 inches to 0.040 inches, more typically the about 0.030 inches thick. In this embodiment, the elastomeric layer provides a compression seal when the surfaces are mated together and allows for minor defects in the molding of the plates. A suitable elastomer is the same elastomer as used for the valves. The elastomer is injection molded.

In the cassette of the present invention, the upper plate is affixed to the lower plate by an adhesive or by ultrasonic welding. Preferred adhesives are UV curable. Typically, the UV curable adhesive has a viscosity in the range of 1,000 to about 3,500 cps. A suitable UV curable adhesive is MD® Medical Device Adhesive No. 1193-M having a viscosity of about 2750 cps.

In one embodiment of the cassette of the present invention, the 1 to 24 parallel digestion chambers are connected by a respective set of 1 to 24 parallel channels to their own respective well and buffer loading port on the gel slab positioned in the separation chamber between the top plate and the opposing bottom plate. In another embodiment of the cassette of the present invention, the 1 to 24 parallel digestion chambers are connected respectively by 1 to 24 parallel channels to their respective loading port on 1 to 24 parallel separation chambers.

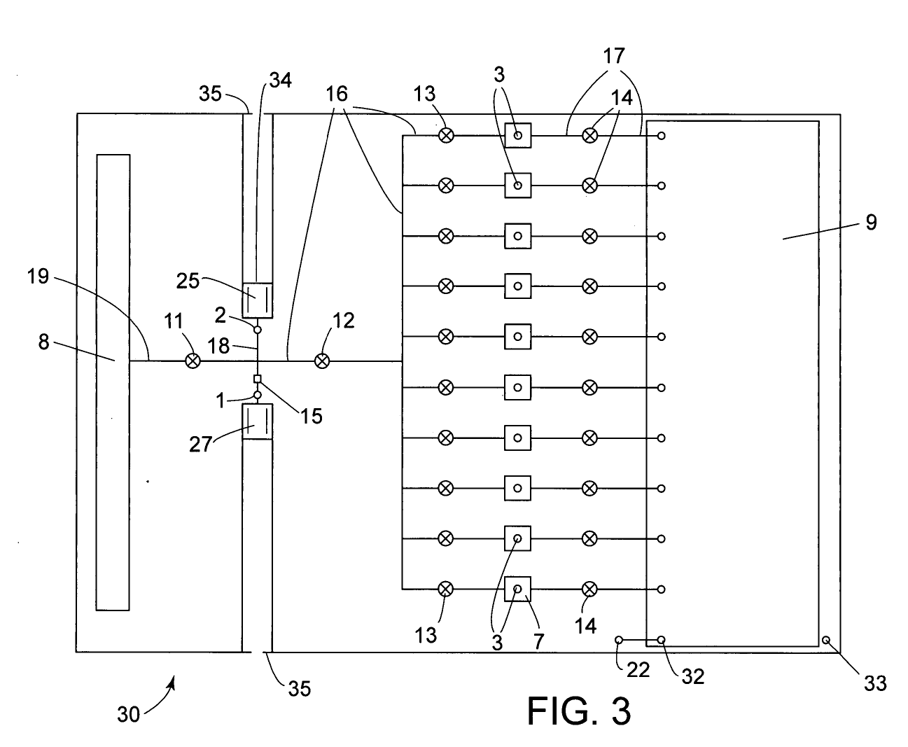

In another embodiment of the cassette of the present invention, both the isolation chamber and the mixing chamber have a piston or plunger moveably sealed therein for drawing fluid therein or pushing fluid thereout or both. See FIG. 2. Preferably, the isolation and mixing chambers are cylindrical. It is convenient if the isolation and mixing chambers are sized so that a plunger tip from a LUER® insulin syringe (1 cc) can be used as the piston or plunger. In this embodiment, the distal end of the plungers form a moveable wall in the isolation chamber and in the mixing chamber. In one embodiment, the plunger can be provided without a piston shaft as shown in FIG. 3. In an alternative embodiment, only the plunger tip is provided in the cassette, and the proximal end of the plunger tip has its standard recess for receiving and capturing the tip of the plunger shaft. Restrictors 35, shown at the outer (distal) end of the isolation chamber and at the outer end the mixing chamber, retain the plunger tips in the chambers, thereby preventing any contents from within the chambers from escaping beyond the walls of the cassette. Preferably, the restrictor is placed further within the chamber to limit to volume of sample or reagent or both capable of being drawn within the chamber.

The cassette of the present invention can be better understood reference to the figures. FIG. 1 discloses a schematic of a liquid flow system 10, for inclusion in a cassette of the present invention, including channels (16, 17, 18 and 19) and chambers (5, 6, 7, 8 and 9). In this schematic, a sample receiving port 1 is in fluid communication with an isolation chamber 5. A diluent receiving port 2 is in fluid communication with a mixing chamber 6. The isolation chamber 5 is in fluid communication, via channel 18, with the mixing chamber 6. Channel 18 has a filter 15 therein of sufficiently small pore size for retaining cells and cell debris in the isolation chamber. The isolation chamber 5 and the mixing chamber 6 are isolated from the remainder of the system 10 by valves 11, 12 and/or 13. The isolation chamber 5 and the mixing chamber 6 are connected to waste chamber 8 via first channel 19. The isolation chamber 5 and the mixing chamber 6 are connected to the reaction chamber 7 via second channel 16. In the embodiment shown, the isolation chamber 5 and the mixing chamber 6 are isolated from the reaction chamber by valves 12 and 13 in channel 16. However, it is sufficient to employ only the parallel series of valves 13 to separate the isolation chamber 5 and the mixing chamber 6 from the reaction chamber. The function of valve 12 is to minimize any sample from entering channel 16 before being fully processed. The series of valves 13 operate independently or in unison in response to a triggering action outside the cassette. When valves 12 and 13 are open and valve 11 is closed, the application of a controlled amount of positive pressure to the contents of chambers 5 or 6 would drive the contents of one of those chambers to the one or more reaction chambers 7. By controlling the volume of pressure applied and the valving, precise amounts of processed sample could be transferred into each reaction chamber. Positive pressure is applied to the contents of chambers 5 and 6 by compressed air or liquid or by the use of pistons (as shown in FIG. 2). The compression of a piston 27 would drive any processed sample from the respective chamber (5 or 6) to the one or more of the reaction chambers 7. The reaction chamber 7 has a port 3 for receiving reagents for reacting with any sample transported thereto. The reaction chamber 7 is also capable of functioning as an amplification chamber and as a digestion chamber when the sample is DNA. The reaction chamber 7 is in fluid communication with the separation chamber 9, which includes in one embodiment a gel slab (not shown) for electrophoresis, or in another embodiment, a capillary electrophoresis tube for each reaction chamber.

FIG. 2 discloses a flow system 20, including channels (16, 17, 18 and 19) and chambers (5, 6, 7, 8 and 9), of one embodiment of the cassette of the present invention. In this schematic, a sample receiving port 1 is in fluid communication with an isolation chamber 5. A diluent receiving port 2 is in fluid communication with a mixing chamber 6. The isolation chamber 5 is in fluid communication, via channel 18, with the mixing chamber 6. Isolation chamber 5 has a piston 27, attached to a piston shaft 26, for drawing sample, lysing reagent, diluent and/or reagents therein. The piston 27 is shown in a partially open position, wherein the distal wall 23 of the piston forms the moveable outside wall of isolation chamber 5. Mixing chamber 6 has a piston 25, attached to a piston shaft 24, for drawing diluted sample, diluent and reagents therein. Channel 18 has a filter 15 therein of sufficiently small pore size for retaining cells and cell debris in the isolation chamber. The isolation chamber 5 and the mixing chamber 6 are isolated from the remainder of the system 10 by valves 11, 12 and/or 13. The isolation chamber 5 and the mixing chamber 6 are connected to a waste outflow 28 via first channel 19. The waste outflow 28 may connect to a waste chamber 8 (shown in FIG. 1) or to a line outside the cassette that would remove waste and transport it if away from the cassette. The isolation chamber 5 and the mixing chamber 6 are connected to the reaction chamber 7 via second channel 16. In the embodiment shown, the isolation chamber 5 and the mixing chamber 6 are isolated from the series of reaction chambers 7 (shown in parallel) by valves 12 and 13 in second channel 16. However, it is sufficient to employ only the parallel series of valves 13 to separate the parallel reaction chambers 7 from the isolation chamber 5 and/or the mixing chamber 6. The function of valve 12 is to minimize any sample from entering channel 16 before being fully processed. The series of valves 13 operate independently or in unison in response to a triggering action outside the cassette. When valves 12 and 13 are open and valve 11 is closed, the compression of piston 27 (or 25) would drive any processed sample therein from the respective chamber to the one or more reaction chambers. The reaction chamber 7 has a port 3 for receiving reagents which would react with any sample transported therein. The reaction chamber 7 is also capable of functioning as an amplification chamber and as a digestion chamber when the sample is DNA. The reaction chamber 7 is in fluid communication with a capillary electrophoresis tube for each reaction chamber. Port 29 is in fluid communication with a capillary electrophoresis tube and is suited for loading with a DNA ladder (typically 100 bp to 3,000 bp) when the sample is suspected of containing a target DNA, or with a molecular weight ladder when the sample is suspected of containing a target protein.

FIG. 3 discloses a 10 reaction well cassette 30 of the present invention that allows for amplification of 10 different (or the same) DNA targets from a single biological sample. In cassette 30, a sample receiving port 1 is in fluid communication with an isolation chamber 5 (See FIG. 1). A diluent receiving port 2 is in fluid communication with a mixing chamber 6. The isolation chamber 5 is in fluid communication, via channel 18, with the mixing chamber 6. Isolation chamber 5 has a piston 27 (shown in the closed position) for drawing sample, lysing reagent, diluent and/or reagents therein. Mixing chamber 6 has a piston 25 (shown in the closed position) for drawing diluted sample, diluent and reagents therein. The ends of chambers 5 and 6 each have a restriction member 35 that partially occludes the ends of the chambers and prevents the plungers 25 and 27 from being withdrawn from the cassette. The proximal end 34 of the plunger 34 (and likewise for plunger 27) has a member for receiving and engaging the distal end of a plunger shaft (not shown). In one embodiment of the present invention, the cassette would have the shafts in the plungers. In another embodiment, the plunger shafts would be a part of a device that is separate form the cassette but which would operate the pistons in the cassette in response to some preprogrammed information. Channel 18 has a filter 15 therein of sufficiently small pore size for retaining cells and cell debris in the isolation chamber. The isolation chamber 5 and the mixing chamber 6 are isolated from the remainder of the flow system of the cassette by valves 11, 12 and/or 13. The isolation chamber 5 and the mixing chamber 6 are connected to a waste chamber 8 via first channel 19. The isolation chamber 5 and the mixing chamber 6 are also connected to the reaction chamber 7 via second channel 16. In the embodiment shown, the isolation chamber 5 and the mixing chamber 6 are isolated from the series of ten reaction chambers 7 (shown in parallel) by valves 12 and 13 in second channel 16. However, it is sufficient to employ only the parallel series of valves 13 to separate the parallel reaction chambers 7 from the isolation chamber 5 and/or the mixing chamber 6. The function of valve 12 is to minimize any sample from entering channel 16 before being fully processed. The series of valves 13 operate independently or in unison in response to a triggering action outside the cassette. When valves 12 and 13 are open and valve 11 is closed, the compression of piston 27 (or 25) would drive any processed sample therein from the respective chamber to the one or more reaction chambers. Each of reaction chambers 7 has a port 3 for receiving reagents which would react with any sample transported therein. The reagents provided to each of the reaction chambers 7 can be the same or different. For example, in one embodiment the samples could be run in duplicate in which case two of the reaction wells would be provided with the same reagents. More typically, the reagents are different so that cassette 30 is capable of performing 10 different tests on a single sample. When the sample is suspected of containing a target DNA, the reaction chamber 7 functions as both a digestion chamber and an amplification chamber. The reaction chamber 7 is in fluid communication, via channel 17, with a dedicated sample well 32 for each reaction chamber 7. A series of parallel valves 14 remain closed until the digested DNA product in each reaction chamber is ready for transport to a parallel series of sample wells 32 on the electrophoretic slab gel in the separation chamber 9. The samples are moved from chamber 7 by closing valve 13 and opening the corresponding valve 14 and driving the appropriate aliquot of sample into well 32 by displacement of that volume through port 3. Port 22 is in fluid communication with a dedicated control well 32 in the electrophoretic gel and is suited for loading a DNA ladder and buffer when the sample is suspected of containing a target DNA. Likewise port 22 is suited for loading a molecular weight ladder and a buffer when the sample is suspected of containing a target protein. In cassette 30, there is a series of loading wells 32 in the slab gel in separation chamber 9. Electrodes for the electrophoresis are also provided via ports 22 and 33 respectively. Typically, port 33 would provide access to a reservoir of buffer medium which in turn would be in contact with the distal end of the electrophoretic medium.

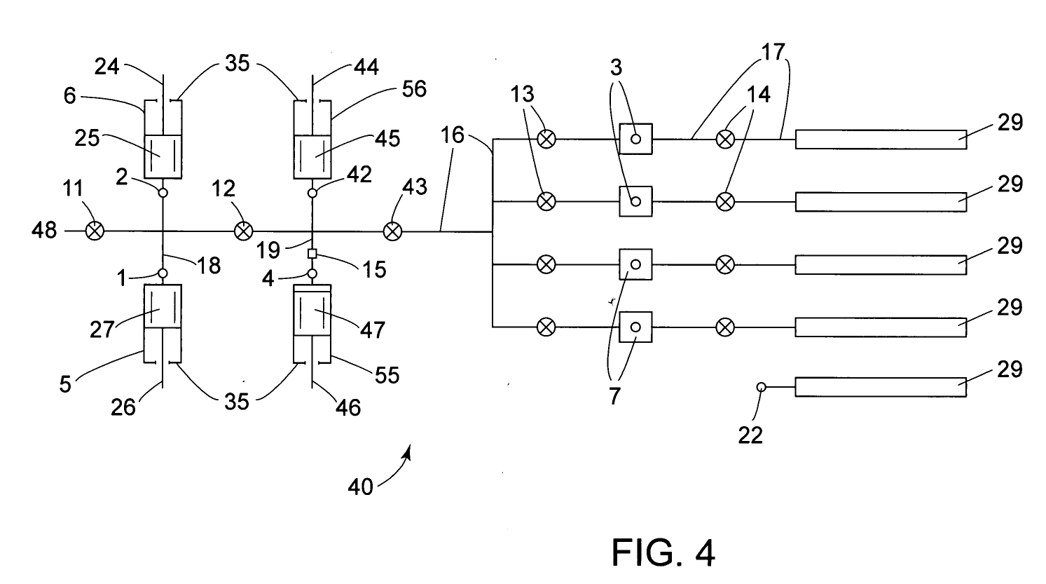

FIG. 4 discloses a schematic of another flow system 40 that is utilized in another embodiment of a cassette of the present invention. This schematic is analogous to that shown in FIG. 2, except that the present schematic provides for two additional mixing chambers for isolating DNA or protein from a biological sample. In the schematic of FIG. 4, a sample receiving port 1 is in fluid communication with an isolation chamber 5. A diluent receiving port 2 is in fluid communication with a mixing chamber 6. The isolation chamber 5 is in fluid communication, via channel 18, with the mixing chamber 6. Isolation chamber 5 has a piston 27 (shown in the closed position) connected to a shaft 26 for drawing sample, lysing reagent, diluent and/or reagents therein. Mixing chamber 6 has a piston 25 (shown in the closed position) connected to shaft 24 for drawing diluted sample, diluent and reagents therein. The ends of chambers 5 and 6 each have a restriction member 35 that partially occludes the ends of the chambers and prevents the plungers 25 and 27 from being withdrawn from the cassette. Channel 18 has a filter 15 therein of sufficiently small pore size for retaining cells and cell debris in the isolation chamber. The isolation chamber 5 and the mixing chamber 6 are isolated from the remainder of the flow system of the cassette by valves 11, 12 and/or 13. The isolation chamber 5 and the mixing chamber 6 are connected to a waste chamber 8. The isolation chamber 5 and the mixing chamber 6 are also connected to the reaction chamber 7 via second channel 16. In the embodiment shown, the isolation chamber 5 and the mixing chamber 6 are isolated from the series of ten reaction chambers 7 (shown in parallel) by valves 12 and 13 in second channel 16. However, it is sufficient to employ only the parallel series of valves 13 to separate the parallel reaction chambers 7 from the isolation chamber 5 and/or the mixing chamber 6. The function of valve 12 is to minimize any sample from entering channel 16 before being fully processed. The series of valves 13 operate independently or in unison in response to a triggering action outside the cassette. When valves 12 and 13 are open and valve 11 is closed, the compression of piston 27 (or 25) would drive any processed sample therein from the respective chamber to the one or more reaction chambers. Each of reaction chambers 7 has a port 3 for receiving reagents which would react with any sample transported therein. The reagents provided to each of the reaction chambers 7 can be the same or different. For example, in one embodiment the samples could be run in duplicate in which case two of the reaction wells would be provided with the same reagents. More typically, the reagents are different so that cassette 30 is capable of performing 10 different tests on a single sample. When the sample is suspected of containing a target DNA, the reaction chamber 7 functions as both a digestion chamber and an amplification chamber. Each reaction chamber 7 is in fluid communication, via channel 17, with a capillary electrophoresis tube 29. The flow of liquid through channel 17 is controlled by valve 14. The samples are moved from reaction chamber 7 by closing valve 13 and opening the corresponding valve 14 and driving the appropriate aliquot of sample into the end of the electrophoretic medium by displacement of that volume through port 3. Port 22 is in fluid communication with its own dedicated capillary electrophoresis tube 29 and is suited for loading a DNA sizing ladder and buffer when the sample is suspected of containing a target DNA. Likewise port 22 is suited for loading a molecular weight ladder and a buffer when the sample is suspected of containing a target protein.

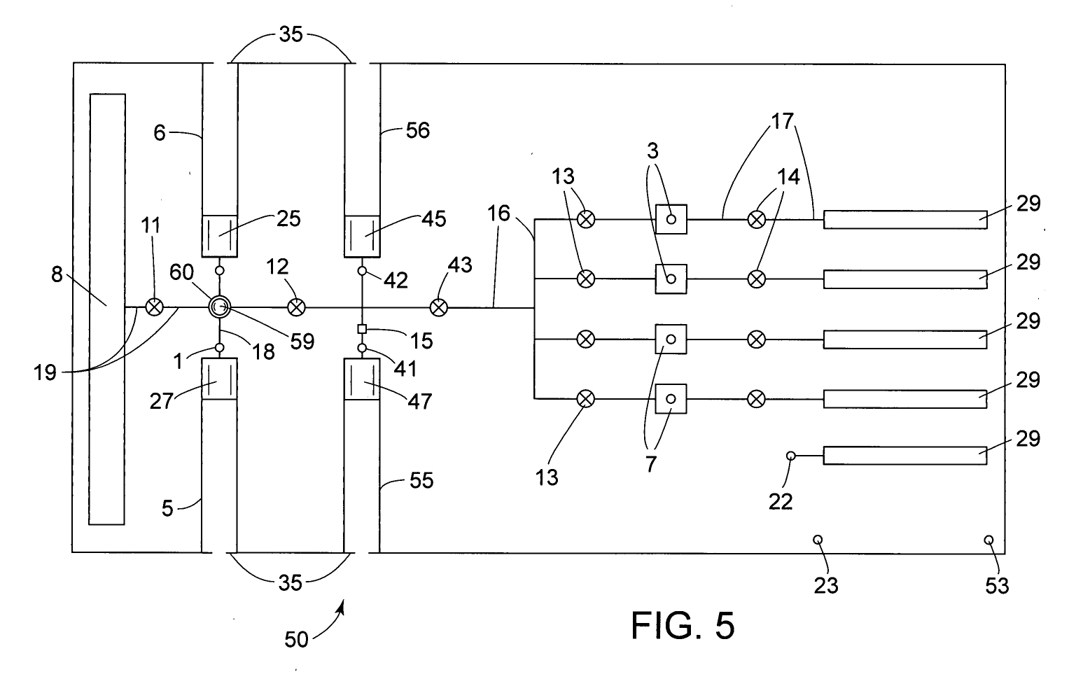

FIG. 5 discloses a cassette 50 of the present invention embodying the flow system of FIG. 4. The cassette 50 includes a port 53 for receiving buffer to contact the distal ends of the electrophoretic capillaries 29 and for receiving the anode. A separate port 23 receives buffer for contacting the proximal end of the capillaries 29 and also receives the cathode.

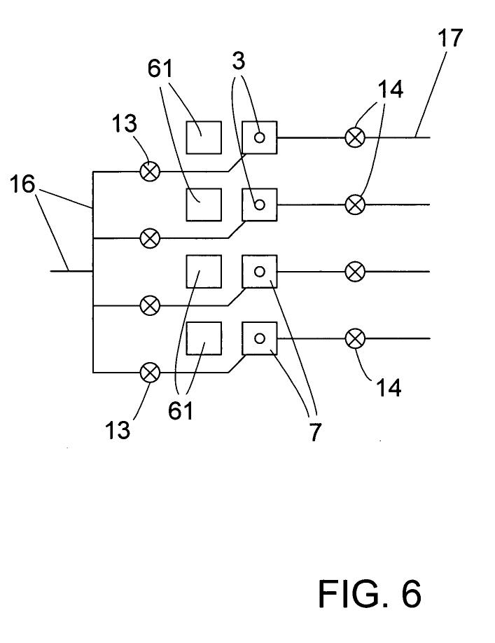

FIG. 6 discloses another embodiment of the cassette of FIG. 5 wherein cavities 61 allow one to monitor the reaction in each of reaction chambers 7 by measuring the fluorescence in cavities 61 when the reaction solution in the reaction chambers is stimulated with an excitation frequency. Specifically, the plastic used in the cassette of FIG. 5 is UV transmissible. Cavities 61 are open and suitably sized for receiving a mirror that reflects any fluorescence or absorbance by the contents of the reaction chamber of the cassette to an appropriate detector positioned outside the cassette. The mirror can be positioned at any angle that would reflect signal to an appropriate detector. Typically, the external mirror is positioned at a 45° angle to the side wall of the cavity (and the side wall of the aligned reaction well), thereby reflecting the signal in the upward or downward direction to the appropriately positioned detection apparatus. Thus, when an excitation beam is directed into a reaction chamber, any fluorescence associated with the formation of the reaction product would be scattered in all direction, including into the cavity 61 where it would be reflected by the external mirror to an external sensor for monitoring of the reaction. The use of fluorescent labels to monitor a DNA (or PCR) amplification reaction is well known in the art.

Ethidium bromide has been used for many years to visualize the size distribution of nucleic acids separated by gel electrophoresis. The gel is usually transilluminated with ultraviolet light and the red fluorescence of double stranded nucleic acid observed. Specifically, ethidium bromide is commonly used to analyze the products of PCR after amplification is completed. Furthermore, EPA 0 640 828 A1 to Higuchi & Watson, hereby incorporated by reference, discloses using ethidium bromide during amplification to monitor the amount of double stranded DNA by measuring the fluorescence each cycle. The fluorescence intensity is noted to rise and fall inversely with temperature was greatest at the annealing/extension temperature (50° C.), and least at the denaturation temperature (94° C.). Maximal fluorescence is acquired each cycle as a measure of DNA amount.

U.S. Pat. No. 6,569,627 which issued on May 27, 2003 to Wittwer, et al., entitled “Monitoring hybridization during PCR using SYBR Green I,” and incorporated herein by reference, discloses a method of real time monitoring of a polymerase chain reaction amplification of a target nucleic acid sequence in a biological sample which comprises: (a) adding to the biological sample an effective amount of two nucleic acid primers and a nucleic acid probe, wherein one of the primers and the probe are each labeled with one member of a fluorescence energy transfer pair comprising an acceptor fluorophore and a donor fluorophore, and wherein the labeled probe hybridizes to an amplified copy of the target nucleic acid sequence within 15 nucleotides of the labeled primer; (b) amplifying the target nucleic acid sequence by polymerase chain reaction; (c) illuminating the biological sample with light of a selected wavelength that is absorbed by said donor fluorophore; and (d) detecting the fluorescence emission of the sample.

U.S. Pat. No. 6,258,569, which issued on Jul. 10, 2001 to Livak, et al., entitled “Hybridization assay using self-quenching fluorescence probe,” and incorporated herein by reference in its entirety, discloses a method of monitoring nucleic acid amplification on a target polynucleotide using a nucleic acid polymerase having 5′ to 3′ nuclease activity, a primer capable of hybridizing to the target polynucleotide, and an oligonucleotide probe under amplification conditions wherein the probe hybridizes to the target polynucleotide 3′ relative to the primer and the probe does not hybridize with itself to form a hairpin structure. The oligonucleotide probe has at one end a fluorescent reporter and at the other end a quencher that quenches the fluorescence of the reporter molecule when both the fluorescent reporter and quencher are attached to the probe. Digestion of the oligonucleotide probe by the polymerase during amplification is effective to separate the reporter from the quencher, whereby a fluorescence signal of the reporter is increased.

FIG. 7 discloses the flow system of FIG. 1 wherein an elastomeric strip 71 over the channels functions as a valve array (for valves 13 and 14) at the points where the strip crosses the channel. Likewise, elastomeric strip 72, which is dogbone shaped, provides valves 11 and 12 at the heads of the dogbone. The valve consists of a hole or pore in the plate of the cassette that connects the face on the elastomer to the channel in the cassette. In this embodiment, the valves are open in the default position as shown. The valves are closed when a pin or probe pushes the elastomer into the hole or pore and into the channel so as to completely obstruct to flow of liquid in the channel. Release of the pins causes the elastomer to pull out of the channel and hole, reopening the channel.

### EXAMPLE 1

Making a Cassette of the Present Invention

The bottom plate of the cassette comprised of a UV transparent acrylic was placed face up on solid surface. A silk screen assembly manufactured by AsahiTec America, Richmond, Ind. and modified to align with the bottom plate was positioned over the bottom plate and suspended ˜1-2 mm above the bottom plate. The silk screen was 330-mesh polyester; 35μ thread diameter; 42μ mesh opening and had a 56±3μ overall thickness. A bead of U.V.-curable adhesive was manually applied directly to the silk screen (i.e., above the silk screen pattern). A squeegee (#CPS-5070 with wood handle, 70-75 durometer (Shore ‘A’ scale), A.W.T. World Trade, Inc., Chicago, Ill.) with square-edge tip was dipped into the adhesive bead on the silk screen (see above). Starting at the top of silk screen pattern, adhesive was applied to the silk screen pattern with the squeegee tip at 45-degree angle (to the perpendicular) while maintaining constant squeegee pressure and application speed. The silk screen assembly was removed from the bottom plate of the cassette. The plungers and filter were positioned in appropriate locations on the bottom plate. Any gel slab or capillaries for electrophoresis are positioned in the separation chamber. The top plate of the cassette was brought into contact with the bottom plate while maintaining part alignment. The resulting cassette sandwich was placed in the nest of a custom press (Buck Enterprises, St. Charles, Ill.). Pressure (300-500 psi) was applied to the cassette sandwich and it was exposed for approximately 60 seconds to a U.V. light source [Model 5000 AS modular lamp with mounting stand kit, Dymax Corp., Torrington, Conn.; typical intensity in UVA range (320-390 nm) at 3″ from lower edge of reflecting range: 225 mW/cm²)] which was positioned the below the press containing the cassette. After 60 seconds, the UV light was turned off, the pressure released, and the assembled cassette was removed from the press.

Addition of the Elastomer Valves:

The elastomer valves were affixed to the bottom plate of the cassette by injection molding. When the elastomer valves are elastomeric strips positioned between the plates, the strips must be put on by injection molding before the cassette is sealed. When the elastomer valves are elastomeric strips that are positioned on the outside face of the top plate or the bottom plate, the strips may be placed on the cassette by injection molding either before or after the cassette is sealed. Preferably, the valves are molded on the outside face of the cassette before the two opposing plates are mated and sealed.

### EXAMPLE 2

DNA Assay Using the Four Mixing Chamber Embodiment (FIG. 5) of the Cassette of the Present Invention

The cassette of FIG. 5 is removed from its sterile pouch and placed into the nest of an appropriate instrument. Valves 11, 12 and 43 are placed in the closed position. Pistons 25, 27, 45 and 47 are in the closed (home) positions. Four piston shafts engage the proximal ends of the four pistons, respectively, and seat therein. A biological sample (100 μL) containing nucleated cells suspected of containing target DNA is injected from a sample syringe into a first mixing chamber (MC) 5 through sample port (SP) 1. The piston 27 in MC 5 is retracted sufficiently to intake the 100 μL of sample. Lysis solution (50 μL) from reagent syringe #1 was then added to MC 5 via SP 1. The piston 27 in MC 5 is retracted sufficiently to intake the 50 μL of lysis solution. Mixing of both solutions was achieved by repeated transfers (cycling 3×) of the total volume between MC 5 and MC 6 using pistons 27 and 25 to drive the solution back and forth. In addition to chemical lysis of cells, physical disruption of the cells was effected by passage of the solution by a polystyrene stearate bead 59 positioned in a slightly larger circular chamber 60 between both mixing chambers MC 5 and MC 6. With all of the lysate in MC 5, valve 12 is opened and 150 μL of the resulting lysate was transferred from MC 5 to MC 55. To affect the transfer, piston 27 in MC 5 is closed and piston 47 in MC 55 is opened sufficiently to receive the 150 μL of lysate. Any cell debris greater in size than 1 μm is retained outside of MC 55 by a 1 μm pore size nylon filter 15 positioned in the channel outside of MC 55. A suspension (25 μL) of 3-5 μm latex (or alternate material) microparticles in a high salt concentration is injected from a reagent syringe into MC 55 via SP 41. Simultaneously, piston 47 is retracted a sufficient amount to intake the 25 μL of suspended microparticles. The genomic DNA in the lysate is captured by the microparticles as the two solutions are mixed by repeated transfers (cycling 2-3×) of the lysate between MC 55 and MC 56. During cycling, the microparticles are retained in MC 55 by the 1 μm pore size nylon filter 15 in the channel from MC 55 to MC 56. With valves 11 and 12 in the open position, piston 47 is brought to the ‘home’ position which concentrates the microparticles against the nylon filter and transfers unbound material, including protein and cell debris, to the waste reservoir (WR) 8. With valve 12 closed, wash buffer (175 μL) from reagent syringe #3 is injected into MC 56 via SP 42, with piston 45 simultaneously being retracted a sufficient amount to intake the wash solution into MC 56. Piston 45 closes while piston 47 is retracted a corresponding amount to transfer the total wash solution into MC 55, thereby resuspending the concentrated microparticle/DNA complex and washing it. The wash solution is cycled 2× between MC 55 and MC 56. The wash solution from MC 55 is transferred to the waste reservoir 8 by opening valves 12 and 11 and closing piston 47. Washing of the microparticles is repeated one more time as described above. Elution buffer (200 μL) is injected from reagent syringe #4 to MC 56. The elution buffer volume is then cycled (2×) between MC 56 and MC 55 to effect elution of DNA from the microparticles. The isolated and purified genomic DNA in MC 55 is transferred (18 μL) sequentially to each of the reaction chambers (RC) 7. Specifically, valve 12 closes and valves 43 and 13 are opened. Piston 47 compresses the fluid in chamber 55 to drive an 18 μL aliquot in each of reaction chambers 7 by the sequential opening and closing of valves 13 in series until each reaction chamber 7 receives its precise amount of sample. With valves 13 and 14 closed, a concentrated amplification mix (5 μL) is injected from reagent syringe #5 into each RC via the corresponding reagent port 3. For isothermal amplification, the reaction mixture is allowed to proceed for between about 30 min to 2 hours at 37° C. For PCR amplification, the repeated heating and cooling steps (optimized for the specific assay) are performed on the reaction mixtures in each RC via the onboard Peltier device in contact with the cassette bottom. The target DNA that has been specifically amplified (amplicons) is then digested by appropriate restriction enzyme(s) by the addition of 10 μL of buffered restriction enzyme to each of the chambers. The mixtures containing the restriction enzyme are incubated at 37° C. for 20-30 min. Once the time for digestion has passed, the DNA fragments are labeled by adding a solution of art accepted label into the reaction chambers 7. After allowing for mixing, valve 14 is opened, and the DNA digests (e.g., 1-50 μL each) are transferred to the corresponding sample wells of a bufferless 2% agarose slab gel (not shown) or to individual capillary gels 29 within the cassette. The samples are moved by closing valve 13 and opening the corresponding valve 14 and driving the appropriate aliquot of sample into well 32 by displacement of that volume through port 3. A 5 μL aliquot of a solution containing a DNA ladder is introduced into the sample port 22 of a dedicated capillary 29. Buffer and electrodes are then introduced in to ports 22 and 53 at opposing ends of the capillary devices. Electrophoresis of the DNA digests is conducted at 50 mA for 20 min to about 4 hours depending upon the sample size and the size and type of the electrophoretic gel. Following electrophoresis, the separated DNA fragments are visualized and an image of the gel is acquired by the camera positioned above the gel and archived in the instrument. The used cassette and contained contents are safely disposed in a container for contaminated waste.

### EXAMPLE 3

Protein Assay Using the Two Mixing Chamber Embodiment (FIG. 3) of the Cassette of the Present Invention

The cassette of FIG. 3 is removed from its sterile pouch and placed into the nest of an appropriate instrument. Valves 11 and 12 are placed in the closed position. Pistons 25 and 27 are in the closed (home) positions. Two piston shafts engage the proximal ends of the four pistons, respectively, and seat therein. A biological sample (100 μL) is injected from the sample syringe (located on instrument carousel) into a mixing chamber (MC) 5 through sample port (SP) 1 while the instrument simultaneously withdraws piston 27 of MC 5 a corresponding amount to intake the volume of sample. Preferably, the biological sample is prepared to be debris and stroma free outside the cassette. A reagent dispenser outside the cassette dispenses into SP 1 75 μL of a suspension of latex beads (diameter of 2 microns or greater) having a target specific antibody thereon. Simultaneously, piston 27 withdraws the corresponding amount to intake the suspension into MC 5 where it mixes with the protein in the sample. The target protein is captured by the antibodies on the microparticles as the two solutions are mixed by repeated transfers (cycling 2-3×) of the mixture between MC 5 and MC 6. During cycling, the microparticles are retained in MC 5 by the 1 μm pore size nylon filter 15 in the channel from MC 5 to MC 6. With valve 11 in the open position, piston 27 is brought to the ‘home’ position which concentrates the microparticles against the nylon filter and transfers unbound material, including non-target protein to the waste reservoir (WR) 8. With valves 11 and 12 closed, wash buffer (175 μL) from reagent syringe #3 is injected into MC 6 via SP 2, with piston 25 simultaneously being retracted a sufficient amount to intake the wash solution into MC 6. Piston 25 closes while piston 27 is retracted a corresponding amount to transfer the total wash solution into MC 5, thereby resuspending the concentrated microparticle/target protein complex and washing it. The wash solution is cycled 2× between MC 5 and MC 6. The wash solution from MC 5 is transferred to the waste reservoir 8 by opening valve 11 and closing piston 27. Washing of the microparticles is repeated one more time as described above. Elution buffer (200 μL) is injected from reagent syringe #4 to MC 6. The elution buffer volume is then cycled (2×) between MC 6 and MC 5 to effect elution of the target protein from the microparticles. Valves 12 and 13 are opened and piston 27 is compressed to transfer the isolated target protein from chamber 5 to the reaction chambers 7. Alternately, if the isolated protein solution is contained in MC 6, valves 12 and 13 are opened and piston 25 is compressed to transfer the isolated target protein from chamber 6 to the reaction chambers 7. In either instance, the isolated and purified target protein is transferred (18 μL) sequentially to each of the reaction chambers 7. To characterize the protein, different reagents can be added to each of the reaction chambers via their respective opening 3. Specifically, a first chamber receives 5 μL of an electrophoretic buffer (control). The second chamber receives a detergent, such as sodium dodecyl sulfate, in the buffer to break up any non-covalently bound subunits. The third reaction chamber receives a sulfhydryl agent, such as mercaptoethanol, in the buffer to break up any sulfhydryl bonds, including sulfhydryl bonded subunits. The treated samples in each reaction chamber 7 are sequentially added to a respective well 32 on a gel in the separation chamber 9. The samples are moved by closing valve 13 and opening the corresponding valve 14 and driving the appropriate aliquot of sample into well 32 by displacement of that volume through port 3. Port 22 receives 5 μL of a solution of molecular weight standards followed by an excess of electrophoretic buffer. Ports 22 and 33 are filled with electrophoretic buffer and the corresponding electrode for the electrophoresis. The electrophoresis for 10 minutes to 2 hours, depending upon the sample. The protein on the electrophoretic gel is visualized using standard techniques known in the electrophoretic art. Once the protein is visualized and an image of the gel is acquired by the camera positioned above the gel and archived. The used cassette and contained contents are safely disposed in a container for contaminated waste.

Therefore, it is intended that the invention not be limited to the particular embodiment disclosed, but that the invention will include all embodiments falling within the scope of the appended claims.

1. A plastic cassette suitable for DNA analysis comprising a top plate and an opposing bottom plate affixed thereto, the plates in combination forming therebetween an isolation chamber suitable for isolating DNA from the biological sample suspected of containing the target DNA, one or more reaction chambers in fluid communication with the isolation chamber and suitable for amplifying any target DNA found in the isolation chamber, a digestion chamber which is the same as or in fluid communication with the amplification chamber and suitable for digesting the amplified target DNA (“amplicons”) with restriction endonucleases to produce digestion fragments that are characteristic for the target DNA, and a separation chamber in fluid communication with the digestion chamber for receiving and separating the digested and un-digested amplicons, one of the plates having a port for receiving a biological sample suspected of containing a target DNA, the port being in fluid communication with the isolation chamber.

2. The cassette of claim 1, further comprising a waste chamber for receiving undesired sample, or components or reagents.

3. The cassette of claim 2, wherein the waste chamber is in fluid communication with the isolation chamber by a first channel.

4. The cassette of claim 3, wherein the first channel has a first valve operatively positioned therein.

5. The cassette of claim 1, wherein the isolation chamber is associated with a piston suitable for drawing fluid into the chamber.

6. The cassette of claim 1, wherein the isolation chamber is in fluid communication with the one or more reaction chambers by a second channel.

7. The cassette of claim 1, wherein the second channel has a second valve operatively positioned therein.

8. The cassette of claim 1, wherein the reaction chamber suitable for amplifying any target DNA and the digestion chamber suitable for digesting the target DNA are the same chamber.

9. The cassette of claim 1, wherein the reaction chamber is in fluid communication with the separation chamber by a third channel.

10. The cassette of claim 9, wherein the third channel has a third valve operatively positioned therein.

11. The cassette of claim 1, wherein the separation chamber has a separation medium therein.

12. The cassette of claim 11, wherein the separation medium is an electrophoretic medium.

13. The cassette of claim 12, wherein the electrophoretic medium is a slab of gel or in a capillary.

14. The cassette of claim 13, wherein a portion of the separation chamber is sufficiently transparent for detecting separated restriction fragments therein.

15. The cassette of claim 14, wherein the sufficiently transparent portion of the separation chamber is sufficiently transparent to visible light.

16. The cassette of claim 14, wherein the sufficiently transparent portion of the separation chamber is sufficiently transparent to ultra-violet light.

17. The cassette of claim 16, wherein the separation chamber is about 90% transmissible to UV light.

18. The cassette of claim 16, wherein the separation chamber is about 95% transmissible to UV light.

19. The cassette of claim 16, wherein the separation chamber is about 97% transmissible to UV light.

20. The cassette of claim 19, wherein the plastic is an acrylic.

21. The cassette of claim 20, wherein the acrylic is a polymethylmethacrylate.

22. The cassette of claim 1, wherein the top plate and the opposing bottom plate are injection molded.

23. The cassette of claim 4, wherein the valve comprises an elastomeric rubber.

24. The cassette of claim 23, wherein the valve comprises a layer of elastomeric rubber positioned between the top plate and the opposing bottom plate.

25. The cassette of claim 24, wherein the valve is closed by external compression thereon.

26. The cassette of claim 24, further comprising an elastomeric layer positioned along the edge of the chambers and channels to provide a watertight seal between the upper plate and the lower plate.

27. The cassette of claim 26, wherein the elastomeric layer is a compression seal.

28. The cassette of claim 27, wherein the upper plate is affixed to the lower plate by an adhesive or by ultrasonic welding.

29. The cassette of claim 28, wherein the upper plate is affixed to the lower plate by an adhesive.

30. The cassette of claim 4, wherein the first valve is a compression valve.

31. The cassette of claim 7, wherein the second valve is a compression valve.

32. The cassette of claim 10, wherein the third valve is a compression valve.

33. The cassette of claim 6, wherein the isolation chamber is connected to 1 to 24 parallel reaction chambers.

34. The cassette of claim 33, wherein the 1 to 24 parallel reaction chambers also function as 1 to 24 parallel digestion chambers, respectively.

35. The cassette of claim 34, wherein the 1 to 24 parallel digestion chambers are respectively connected by a 1 to 24 parallel channels to 1 to 24 parallel separation chambers, respectively.

36. The cassette of claim 34, wherein separate channels fluidly connect each of the 1 to 24 parallel digestion chambers to 1 to 24 application points, respectively, on an electrophoretic gel positioned in the separation chamber.

37. The cassette of claim 36, wherein the gel is a slab gel positioned in the separation chamber between the top plate and the opposing bottom plate affixed thereto.

38. The cassette of claim 37, wherein the slab gel is a polyacrylamide gel.

39. The cassette of claim 1, further comprising a mixing chamber in fluid contact with the separation chamber.

40. The cassette of claim 39, wherein both the isolation chamber and the mixing chamber has a piston or plunger moveably sealed therein for drawing fluid therein and or pushing fluid thereout or both.

41. A plastic cassette suitable for DNA analysis comprising a top plate and an opposing bottom plate affixed thereto, the plates in combination forming therebetween a first chamber suitable for receiving a biological sample suspected of containing a target DNA or protein, one or more reaction chambers in fluid communication with the first chamber and suitable for amplifying any target DNA found in the isolation chamber, said reaction chambers including a digestion chamber which is the same as or in fluid communication with the amplification chamber and suitable for digesting the amplified target DNA (“amplicons”) with restriction endonucleases to produce digestion fragments that are characteristic for the target DNA, the fluid communication to each of said one or more reaction chambers being controlled by a respective valve, each of said reaction chambers having a port for providing access to contents therein.
